(19) United States
(12) Patent Application Publication
(10) Pub. No.: US 2020/0393382 A1
(43) Pub. Date: Dec. 17, 2020

(54) Systems, Devices, and Methods for Soil Optimization

(71) Applicant: Soil Logics LLC, Minneapolis, MN (US)

(72) Inventors: Eline van den Haak, Palo Alto, CA (US); Fréderique van den Haak, Boston, MA (US); Arno van den Haak, Redwood City, CA (US)

(21) Appl. No.: 17/008,028

(22) Filed: Aug. 31, 2020

Related U.S. Application Data
(63) Continuation of application No. PCT/IB2019/051633, filed on Feb. 28, 2019.
(60) Provisional application No. 62/637,189, filed on Mar. 1, 2018.

(51) Int. Cl. G01N 21/77; G01N 33/24; A01C 23/02; A01C 21/00; A01G 25/09

(52) U.S. Cl. CPC G01N 21/7703; G01N 33/24; A01C 23/026; G01N 2033/245; A01G 25/09; A01C 21/007; A01C 21/002

(57) ABSTRACT

A method may include collecting a sample of soil via a soil collection unit. Additionally, the method may include mixing the soil with a liquid to form a colorless solution. Further, the method may include dividing the colorless solution into one or more sub-samples and adding a specific reagent to each sub-sample. Additionally, the method may include analyzing each sub-sample to determine a nutrient value via a soil analysis unit in real-time.

### CROSS-REFERENCE TO RELATED APPLICATIONS

This continuation application claims the benefit of priority from International Application No. PCT/162019/051633 filed on Feb. 28, 2019, which claims the benefit of priority from U.S. Provisional Patent Application No. 62/637,189 filed on Mar. 1, 2018. Both applications are incorporated herein by reference.

### TECHNICAL FIELD

Various aspects of the present disclosure relate generally to systems, devices, and methods for soil optimization. More specifically, the present disclosure relates to systems, devices, and methods for performing soil analysis and/or treatment.

### BACKGROUND

Soil is the unconsolidated mineral or organic material on the surface of the Earth that serves as a natural medium for the growth of land plants. Soil is comprised of various particles including inorganic particles (e.g., small rock fragments, numerous minerals), organic matter (e.g., decayed plants, animal residue), and living organisms (e.g., earth worms, insects, bacteria). The composition or makeup of soil can vary greatly based on human interference and environmental factors. The proportions of a particular soil's constituent parts may be more suited towards growing some varieties of plants, trees, shrubs, and/or grasses, while less preferred for growing others. Indeed, each type of plant (e.g., corn, wheat, soy bean, etc.); tree (e.g., olive tree, etc.), shrub (e.g., grapes, etc.), or grass has varied ideal conditions for growth (e.g., specific mineral or nutrient balance, water content, etc.). In order to maintain the proper nutritional content for a growth of a specific plant type, soil may be analyzed and treated to align the soil's nutritional content with a plant type's needs.

Current analysis is often performed by taking one or more representative soil samples at a common depth over a large area. Such samples are then commonly analyzed in a lab to determine the constituent makeup of the soil. Often roughly ten soil samples are taken over a three hectare field, analyzed in an off-site lab, and the results used to make a recommendation to treat (or not to treat) the entirety of the field. Such a generalization is often inadequate to gauge the true composition of the soil. Indeed, such sparse sampling sizes all at a common depth may fail to address discrepancies in soil components across large areas. Additionally, requiring such samples to be analyzed in a lab slows the responsiveness of soil treatment, if such treatment is ultimately determined to be necessary. Further, if a decision to treat (e.g., fertilize or irrigate) soil is made, typically such treatment includes delivery of a generalized cocktail of numerous nutrients and/or chemicals across the entirety of the sampled field. Such generalized large-scale treatment of soil may result in eutrophication, ground water contamination, insufficient fertilization, and overall energy and scarce resources waste.

The systems, devices, and methods of the current disclosure may address some of the deficiencies described above or address other aspects of the prior art.

### BRIEF SUMMARY

Embodiments of the present disclosure relate to, among other things, systems, devices, and methods for soil optimization. Each of the embodiments disclosed herein may include one or more of the features described in connection with any of the other disclosed embodiments.

In one arrangement a system may include a vehicle including a controller. The system also may include an onboard soil analysis unit including a soil preparation unit and a soil analysis system for real-time analysis.

Examples of the system may include one or more of the following features. The vehicle may be a first vehicle, the system may further include at least one additional vehicle having an onboard analysis unit. Each vehicle may be configured for autonomous operation. The system may include a soil collection unit having a drill and/or soil capturing device. The system may include a fertilization and/or irrigation unit. The fertilization and/or irrigation unit may include a plurality of modular nutrient tanks or one or more containers for solid pellets or powders. The system may include a central platform, the central platform may be wirelessly communicable with the controller of the vehicle. The system may include a plurality of vehicles, where each vehicle of the plurality of vehicles may be in communication with a central platform and may be configured for performing one or more of soil sampling, soil analysis, soil fertilization, and soil irrigation.

In another arrangement, a method may include collecting a sample of soil via a soil collection unit, mixing the soil with a liquid to form a colorless solution, and dividing the colorless solution into one or more sub-samples and adding a specific reagent to each sub-sample. The method also may include analyzing each sub-sample to determine a nutrient value via a soil analysis unit in real-time.

Examples of the method may include one or more of the following features. Dividing the colorless solution into one or more sub-samples may include dividing the colorless solution into eight sub-samples. Analyzing each sub-sample may include taking a photometric measurement of the sub-sample. The method also may include wirelessly delivering the photometric measurement to a central platform and delivering a recommendation to a user. The method also may include executing the recommendation autonomously. Further, the method may include delivering a tailored fertilizer to a field based at least on the photometric measurement. Analyzing each sub-sample and delivering the tailored fertilizer may be done in real-time. The method also may include maneuvering a vehicle coupled to the soil analysis unit autonomously. Collecting a sample of soil via a soil collection unit may include collecting a plurality of samples at varying depths. Mixing the soil with a liquid may include mixing the soil with a Morgan Solution. The method may further include analyzing the data collected via the soil analysis unit, and correlating the data with stored data on a central platform, delivering a recommendation to a user.

In a further example, an analysis unit may include a soil preparation unit, having a mixing chamber, a fluid pump, and a source of Morgan Solution. The analysis unit may further include a soil analysis system having a plurality of reagent tanks. Further, the analysis unit may be coupled to a vehicle.

Examples of the analysis unit may include one or more of the following features. The soil analysis system may further include a plurality of sub-sample paths. The soil analysis system may include at least eight sub-sample paths. The soil preparation unit may further include a mixer configured to mix soil and Morgan Solution to form a colorless solution.

Both the foregoing general description and the following detailed description are exemplary and explanatory only and are not restrictive of the features, as claimed. As used herein, the terms “comprises,” “comprising,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” As used herein, the terms “about,” “substantially,” and “approximately,” indicate a range of values within +/−5% of a stated value.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 illustrates an exemplary soil optimization system;

FIG. 2 is a schematic illustration depicting the flow of soil as it passes through the system of FIG. 1;

FIG. 3 illustrates an exemplary soil collection unit of the system of FIG. 1;

FIG. 4 illustrates a soil preparation unit of the system of FIG. 1;

FIG. 5 is a cross-sectional view of the soil preparation unit of FIG. 4;

FIG. 6 illustrates an alternative soil preparation unit of the system of FIG. 1;

FIG. 7 illustrates an exemplary soil analysis system of the system of FIG. 1; and

FIG. 8 illustrates various communication signals between components of the system of FIG. 1.

### DETAILED DESCRIPTION

Examples of the present disclosure relate to systems, devices, and methods for soil optimization. Reference will now be made in detail to examples of the present disclosure described above and illustrated in the accompanying drawings. Wherever possible, the same reference numbers will be used throughout the drawings to refer to the same or like parts.

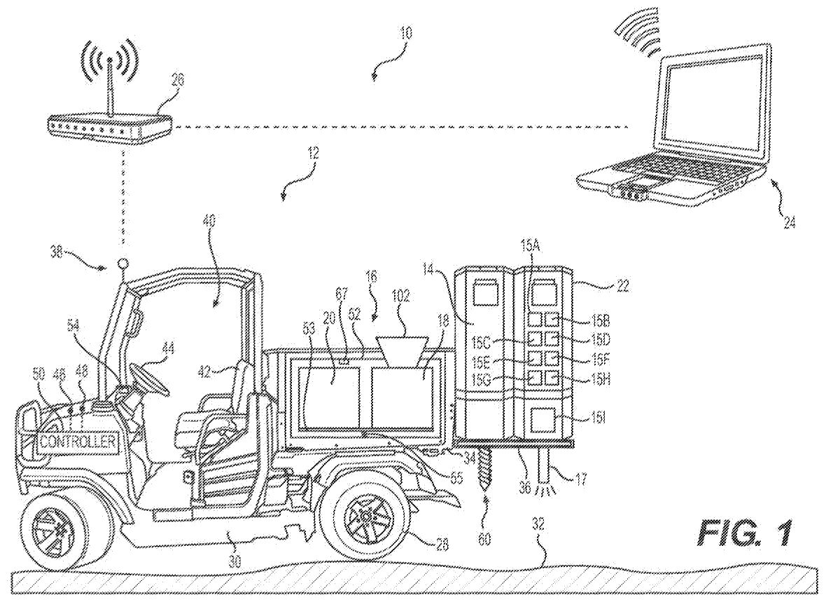

FIG. 1 illustrates an exemplary system 10 for optimization of soil. The system includes a vehicle 12. A soil collection unit 14, an onboard analysis unit 16, and a fertilization/irrigation unit 22 may each be coupled to or housed within vehicle 12. Onboard analysis unit 16 includes a housing 52. Within housing 52, a soil preparation unit 18 and a soil analysis system 20 are coupled to a frame 53 havening a vibration dampening system 55. Vibration dampening system 55 may comprise one or more springs or other such members to absorb or dampen vibration of onboard analysis unit 16 during operation of vehicle 12, as will be described in further detail below.

Fertilization/irrigation unit 22 may contain a plurality refillable tanks or supplies of nutrients 15A-15H, a refillable tank of irrigation fluid (e.g., water) 15I, and an appropriate delivery system which can be an injection device 17 for delivery of such nutrients to soil 32, as will be described in further detail below. Alternatively, refillable tank 15I of irrigation fluid may be replaced with a commercial water maker. Vehicle 12 may communicate with a computer system or central platform 24 via a wireless unit 26. While FIG. 1 only depicts a single vehicle 12, this disclosure is not so limited. Rather, as discussed below, multiple vehicles 12 may cooperate with one another to facilitate one or more functions of system 10 to efficiently optimize soil 32.

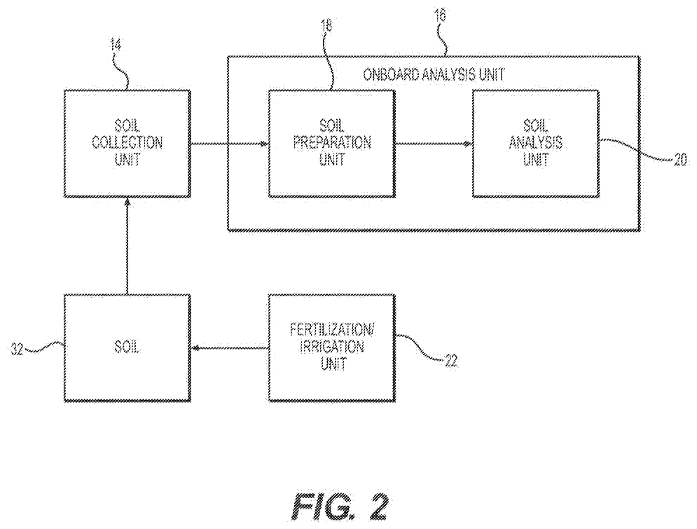

FIG. 2 is a schematic illustration depicting the interaction between soil 32, soil collection unit 14, onboard analysis unit 16, and fertilization/irrigation unit 22. As described in further detail below, soil collection unit 14 may receive a sample of soil 32 therein. Once received, soil collection unit 14 may transfer the sample of soil 32 into onboard analysis unit 16. More particularly, the sample of soil 32 may be delivered to soil preparation unit 18 within onboard analysis unit 16. Alternatively, a user or operator may remove the sample of soil 32 from soil collection unit 14 and deliver the sample of soil 32 into soil preparation unit 18. In so doing, the user may remove any large foreign objects (e.g., large rocks, etc.) before inserting the sample of soil 32 into soil preparation unit 18.

Once received within soil preparation unit 18, the sample of soil 32 may be modified, processed, or otherwise prepared to a form more readily analyzable by soil analysis system 20, e.g., a filtered soil solution. Once preparation of the filtered soil solution via soil preparation unit 18 is complete, the filtered soil solution is delivered (e.g., via gravity or a pump) to soil analysis system 20 for examination. Soil analysis system 20 may divide or partition the filtered soil solution into a plurality of sub-samples. Soil analysis system 20 may test each of the sub-samples of the filtered soil solution to determine a value of one or more nutrients within soil 32. This information may be delivered to central platform 24 via wireless unit 26 (FIG. 1) which then may instruct fertilization/irrigation unit 22 to inject one or more nutrients into soil 32, thereby fertilizing or irrigating soil 32 in real-time. The decision can also be made onboard via controller 50 (described below) without the need for central platform 24. In addition to real-time fertilization and/or irrigation of soil 32, upon receipt of the information to central platform 24, central platform 24 may execute one or more processes (e.g., via a processor) to mine the collected data and prepare a recommendation to add or remove planned sampling and/or analysis activities in real-time.

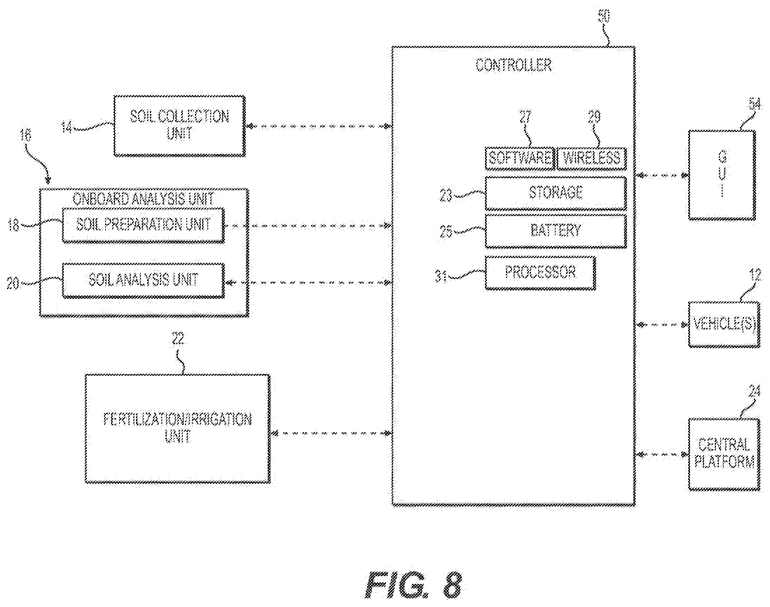

Returning to FIG. 1, vehicle 12 may be any vehicle or machine capable of traversing various field terrains. For example, vehicle 12 may be a Kubota RTV 900 or any other similarly equipped vehicle. Vehicle 12 may include a vehicle propulsion system (e.g., wheels 28 or a continuous track system) coupled to a body 30. Wheels 28 may have sufficient traction and an associated suspension system to enable traversal of various rugged terrains and soils (e.g., soil 32). Vehicle 12 may be designed to meet a range of inclinations and grades and may be weather and dirt-proof or resistant. In some arrangements, vehicle 12 may be powered by one or more of fuels (e.g., gasoline, diesel, formic acid etc.,), an electronic source (e.g., a rechargeable or replaceable battery), a solar panel, and/or fuel cells. Additionally, vehicle 12 may include a controller 50 (schematically illustrated in FIG. 1) containing a processor 31, memory or storage 23, stored software 27, a wireless device 29, and a battery 25, as will be described in further detail in relation to FIG. 8. Controller 50 may receive signals from one or more sensors (e.g., a collision detection/path obstruction sensor 46), a Global Positioning System (“GPS”) tracker 48, onboard analysis unit 16, soil collection unit 14, fertilization/irrigation unit 22, and/or central platform 24 (via wireless unit 26 and a communication unit 38) to determine a location, speed of travel, path obstruction, drive signal, etc. Further, controller 50 may communicate with a man machine interface or graphical user interface 54 (e.g., a screen, monitor, or other such display) of the vehicle 12. User interface 54 may display the progress of one or more activities of system 10, and optionally, may enable operator override functionality. That is, for example, user interface 54 may include one or more buttons (e.g., touch-screen buttons) or the like. If it is determined or desired to alter a pre-planned process (e.g., drilling, fertilization, irrigation), the operator may manipulate the one or more buttons of user interface 54 to change a depth, speed, and/or location of sample collection, fertilization, and/or irrigation. Optionally, such operator override may take place via central platform 24 in addition to or as an alternative to user interface 54.

Interface 54 may enable onboard control of one or more components of system 10. Interface 54 also may display various measurements and data acquired during use. Controller 50 may deliver signals to other vehicles 12 and/or central platform 24. The information collected and/or received may be used by the vehicle's 12 controller 50 to avoid collisions and determine a preferred course of travel, etc.

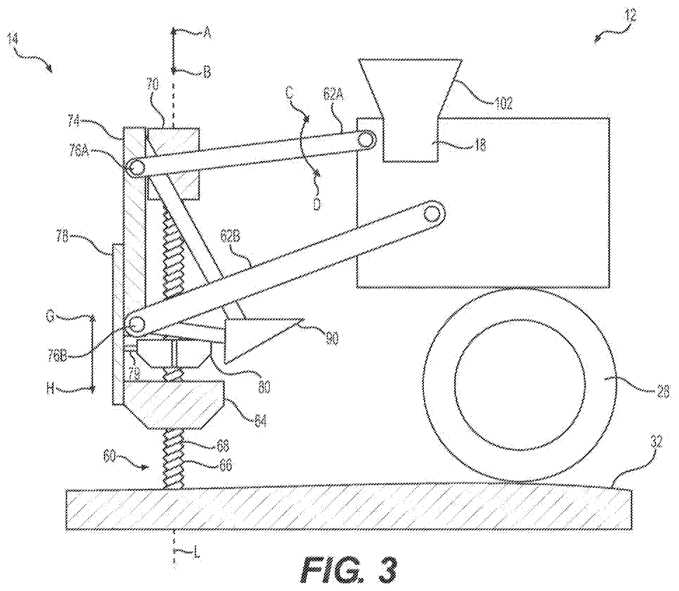

Body 30 may include a bed 34. Bed 34 may support, connect, house, and/or enclose onboard analysis unit 16. Additionally, vehicle 12 may include a frame 36. Frame 36 may be any appropriate device known in the art to mount, connect, or otherwise couple one or more portions (e.g., housings) of soil collection unit 14 and fertilization/irrigation unit 22 to vehicle 12. While soil collection unit 14 is shown as being coupled to vehicle 12 via frame 36, it is understood that at least one or more portions of soil collection unit 14 (e.g., a drill 60, lift 62, and collection tray 64, FIG. 3) may move relative to frame 36 and vehicle 12 so as to adjust a location of drill 60, as will be described in further detail below. Additionally, while fertilization/irrigation unit 22 is shown as being coupled to vehicle 12 via frame 36, it is understood that at least one or more portions of fertilization/irrigation unit 22 (e.g., an injector or sprayer, not shown) may move relative to frame 36 and vehicle 12 so as to adjust a location of the injector or sprayer, as will be described in further detail below.

As shown in FIG. 1, vehicle 12 may include an operator cab 40 including a seat 42 and a steering wheel 44. Additionally, vehicle 12 (or in the case of multiple vehicles, each vehicle 12) may be equipped for autonomous operation. In such a manner, an operator need not steer vehicle 12 via steering wheel 44 in order to operate vehicle 12. For instance, vehicle 12 may be equipped for autonomous driving about a field during sampling and fertilization of the field. To do so, vehicle 12 includes communication unit 38. While communication unit 38 is depicted as located towards a front portion (e.g., in an opposite direction of bed 34) of vehicle 12, such a depiction is merely exemplary. Rather, communication unit 38 may be located anywhere along vehicle 12 so as to facilitate wireless communication to and from central platform 24 (e.g., via wireless unit 26). Additionally, it is understood that communication unit 38 may be arranged for communication (e.g., wireless communication) with controller 50, onboard analysis unit 16, and/or fertilization/irrigation unit 22. Communication unit 38 may be an antenna or similar device capable of receiving and transmitting information (e.g., any appropriate transceiver).

Upon activation of central platform 24, central platform 24 (including a processor and memory having stored software) may deliver commands (e.g., a drive signal) to controller 50 of vehicle 12 (via wireless unit 26 and communication unit 38) to begin driving autonomously or with human interaction. In so doing, central platform 24 also may deliver a path or route for vehicle 12 to travel. The route may take into consideration the size or physical characteristics of the field of soil 32. Such a route may be stored on a memory (not shown) associated with central platform 24. Decision support software stored on the memory associated with central platform 24 may have the ability to optimize and change planned activities and routes based on data received and analysis performed or based on (remote) human intervention e.g., because of inclement weather forecasts.

Alternatively, upon receiving a command to drive, vehicle 12 may determine an appropriate path for traversing the field without the intervention of central platform 24. That is, vehicle 12 may determine an appropriate travel path along the field based on one or more sensors 46 and/or GPS tracker 48, via controller 50 of vehicle 12. Vehicle 12 may develop its own preferred course of travel independent of central platform 24 based on any one or more conditions (e.g., physical characteristics of the field and/or the relative position of additional vehicles 12). For example, if one or more areas of the field is found to contain particularly low (e.g., below or under a stored threshold) levels of nutrients, vehicle 12 may be re-routed to allow for more samples to be taken from the identified low-nutrient level area(s) of the field. Additionally, vehicle 12 may modify a set of commands or pre-determined track based on one or more disturbances. For example, while executing a commanded course of travel, vehicle 12 may encounter an obstruction (e.g., fallen tree, rock, wildlife etc.) unaccounted for in the commanded course of travel. When vehicle 12 senses (e.g., via sensor 46) that there is an obstruction in the path of travel, vehicle 12 may re-route itself to avoid such obstructions. Vehicle 12 may re-route itself according to sensed conditions (e.g., via sensor 46) and/or alternative stored travel paths in a memory or storage 23 located within controller 50 of vehicle 12. Accordingly, vehicle 12 may either operate autonomously to determine its own preferred course of travel, follow commands and/or a pre-determined track, or modify a commanded or pre-determined track to account for obstacles dynamically.

As noted above, system 10 may include a plurality of vehicles 12. Each of the plurality of vehicles 12 may cooperate with one another so as to facilitate soil optimization. Each of the vehicles 12 may include GPS tracker 48, and may coordinate with one another such that none of the vehicles 12 come within a specified distance of one another (e.g., 5 meters) to avoid collisions. Alternatively, two or more of the plurality of vehicles 12 may be physically linked to one another. In either case, vehicles 12 may communicate with one another so as to sequence tasks (e.g., divide the field such that each vehicle 12 analyzes or treats a smaller portion of the field) amongst vehicles 12 to facilitate optimizing soil 32 conditions. Additionally, such sequencing of tasks may be based on decision support analysis and learning, and central platform 24 may assign different tasks to the various vehicles 12, accordingly. Such communication may occur at random or pre-determined times during operation, according to operator programmed specifications and/or process conditions derived in intervals, or upon the sensing of information via sensor 46 and/or location data via GPS tracker 48. Sensor 46 may employ any known sensing technology to determine obstacles or the position of vehicle 12 (or other vehicles 12 located on soil 32). Such sensing technologies may include, by way of example, infrared, echo, tri-dimensional vision systems allowing for three-dimensional vision, collision detection and safety sensors, and tactile sensors.

Soil collection unit 14 may be secured to vehicle 12 in any appropriate location and via any appropriate manner. For example, as shown in FIG. 1, soil collection unit 14 may be coupled to a rear (e.g., end of vehicle 12 opposite driver cab 40). Alternatively, soil collection unit 14 may be coupled to a lateral side surface of vehicle 12 without departing from the scope of this disclosure.

FIG. 3 is a schematic illustration of an exemplary soil collection unit 14. As noted above, soil collection unit 14 includes drill 60 or an alternative scooping device for very shallow samples. Drill 60 may include a thread 66 (e.g., a helical thread) extending along drill 60. Between adjacent windings, crests, or peaks of thread 66 is a root or valley 68. During use, one or more valleys 68 of thread 66 may collect soil 32, as will be described in further detail below. Optionally, drill 60 may be a hollow coring drill bit. In such an arrangements, as drill 60 is activated, soil 32 may enter an internal central hollow passage (not shown) of drill 60, as will be described in further detail below.

In either arrangement, drill 60 may be coupled to vehicle 12 via lift 62. At least one or more portions of soil collection unit 14, e.g., drill 60, lift 62, and collection tray 64, may move relative to vehicle 12 so as to adjust a location of drill 60 and collection tray 64. For example, drill 60 may be arranged for axial and rotational movement and may be operably coupled to a motor 70. Upon activation of a first mode of motor 70, motor 70 may rotate drill 60 to drill into soil 32, thereby collecting soil 32 within valleys 68 and/or an internal central hollow passage of drill 60. Drill 60 may drill into soil 32 to a depth of between about 0 cm and about 60 cm (as measured from the surface of soil 32). Additionally, motor 70 may rotate drill 60 between about 45 and about 100 rpms. Further, it is understood that drill 60 may drill into soil 32 to a variety of depths. Alternatively the unit may be equipped with a range of drills for fixed depths. For example, drill 60 may collect a first sample of soil 32 at a first depth, and one or more additional samples of soil 32 at a different depth.

Additionally, upon activation of a second mode, motor 70 may lift or lower drill 60 along a longitudinal axis L of drill 60, e.g., normal a longitudinal axis of vehicle 12. That is, motor 70 may activate lift 62 to raise drill in the direction of arrow A, or lower drill 60 in the direction of arrow B. Lift 62 may include a linkage system including, for example, links 62A, 62B, support bar 74, and pivots 76A and 76B. For example, motor 70 may be operably coupled to pivot 76A. That is, in the second mode of operation, motor 70 may rotate pivot 76A. Due to the connection of pivot 76A between link 62A and support bar 74, rotation of pivot 76A will result in angular displacement of link 62A relative to support bar 74. For example, rotation of pivot 76A in a first direction (e.g., counter-clock-wise) may rotate link 62A in direction C, shown in FIG. 3. Further, rotation of pivot 76A in a second direction (e.g., clock-wise) may rotate link 62A in direction D, shown in FIG. 3. Accordingly, between sampling locations of the soil 32, drill 60 may be lifted via motor 70 and lift 62. Once lifted, vehicle 12 may navigate to the next sampling location such that drill 60 does not drag along the soil 32, thereby avoiding drill 60 damage.

In order to collect a sample of soil 32 after drilling, collection tray 64 may be raised or lowered in the directions G and H, respectively, as shown, via a collection tray actuator 78, coupled thereto. That is, collection tray actuator 78 may be a motorized lift coupled to support bar 74, moveable with respect thereto. That is, collection tray actuator 78 may slide, translate, or otherwise move linearly along support bar 74 to raise or lower collection tray 64. For example, collection tray 64 may be lowered towards or all the way to the surface of the soil 32, to collect a sample of soil 32 dislodged from drill 60 via a brush 80. For example, brush 80 may be connected to collection tray actuator 78 (via connection 79), for movement therewith. That is, brush 80 may be raised and lowered in the directions G and H, respectively, upon the actuation of collection tray actuator 78. Brush 80 may include one or more bristles (not shown) extending radially inwardly toward drill 60. Such bristles may be directed toward and/or received within the valleys 68 of drill 60. Thus, when drill 60 rotates, the bristles of brush 80 contact soil 32 retained within valleys 68 to thereby dislodge soil 32 from drill 60.

In use, drill 60 may be lowered toward soil 32 via motor 70 and lift 62, as described above. Once lowered, drill 60 may be rotated via motor 70 to drill to a first, initial depth, e.g., about 10 cm. Next, collection tray 64 may be lowered to the surface of soil 32 via collection tray actuator 78. Additionally, drill 60 may be rotated via motor 70 to continue drilling to a preferred final depth, e.g., about 60 cm. Next, drill 60 may be rotated via motor 70 in an opposition direction so as to back out or reverse drill 60, and allow lift 62 to raise drill 60. During or after raising of drill 60, interaction between brush 80 and drill 60 will dislodge soil 32 in valleys 68 and allowed to drop into collection tray 64. Additionally, in an arrangement in which drill 60 includes a hollow coring drill bit, a piston, tube, or other such member (not shown) within drill 60 may actuated to dislodge or push soil 32 collected within the internal central hollow passage (not shown) of drill 60 outwardly thereof toward collection tray 64. Such actuation may occur in any appropriate manner, such as, e.g., via a third mode of motor 70 or a separate actuating mechanism, not shown. Such a separate actuating mechanism may include a pneumatic actuator (e.g., air pressure), a hydraulic actuator (e.g., flush with water), and/or a mechanical actuator (e.g., push rod or relative movement arrangements).

Once the sample of soil 32 has been collected in collection tray 64, the collected sample of soil 32 may be delivered to soil preparation unit 18, either autonomously or via an operator. For example, one or more components of soil collection unit 14 may automatically deliver the collected soil 32 into a chute 102 of soil preparation unit 18 (e.g., via one or more moveable linkages, motor 70, or other such actuation mechanism). Alternatively, a user or operator may remove the sample of soil 32 from collection tray 64 and manually insert the sample through chute 102 of soil preparation unit 18. In order to facilitate entry of the collected soil 32 into soil preparation unit 18, sample filter/chute 90 may be directed to guide the collected soil 32 into soil preparation unit 18. For example, one or more linkages may be actuated to tilt drill 60 approximately 90° from the generally vertical arrangement depicted in FIG. 3 towards a generally horizontal arrangement, not shown. Once tilted, filter/chute 90 may be aligned next to chute 102 such that soil 32 within collection tray 64 may be passed into chute 102 via filter/chute 90. Following collection of a sample of soil 32 via soil collection unit 14, one or more components of soil collection unit 14 may be cleaned in any appropriate manner. For example, drill 60 may be pressure washed to remove built up soil 32 to avoid cross-contamination between samples.

Soil preparation unit 18 may prepare collected samples of soil 32 for soil analysis system 16. That is, soil preparation unit 18 may modify or process samples of soil 32 received from soil collection unit 14 into a form easily or readily analyzed by soil analysis system 20. As noted above, soil preparation unit 18 (as well as soil analysis system 20) may be housed within housing 52 (FIG. 1) of onboard analysis unit 16. Housing 52 may include thermal isolation (e.g., any appropriate thermally insulating material). That is, housing 52 may prevent materials (e.g., soil or chemical solutions therein) from deviating from a desired temperature or temperature range so as to control temperature-dependent and/or temperature-sensitive materials from inadvertently reacting. A temperature sensor 67 may be located within onboard analysis unit 16 (e.g., within housing 52), and may send a signal indicative of a temperature within onboard analysis unit 16 to controller 50, as will be described in further detail below.

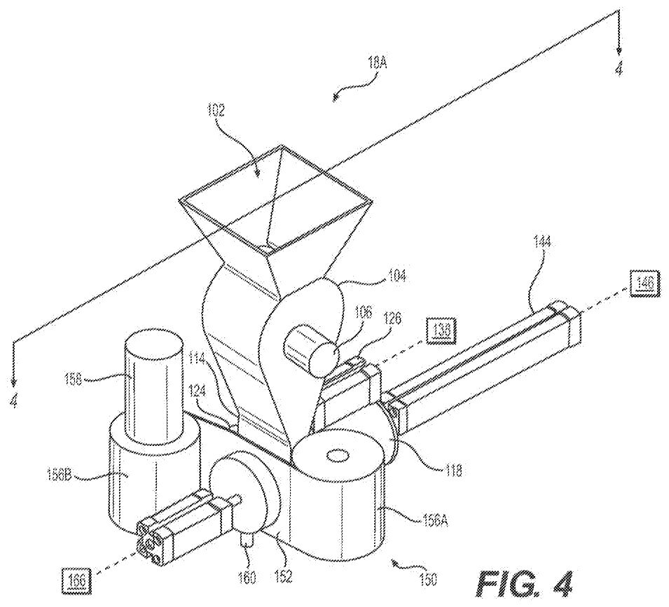

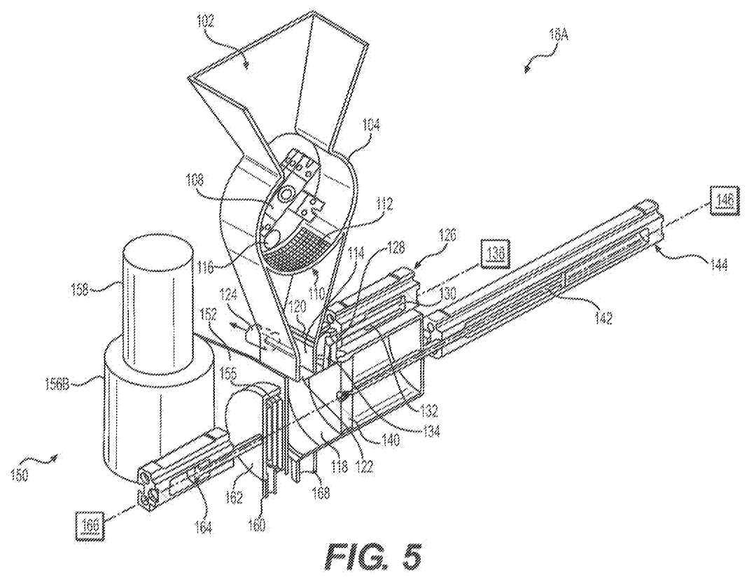

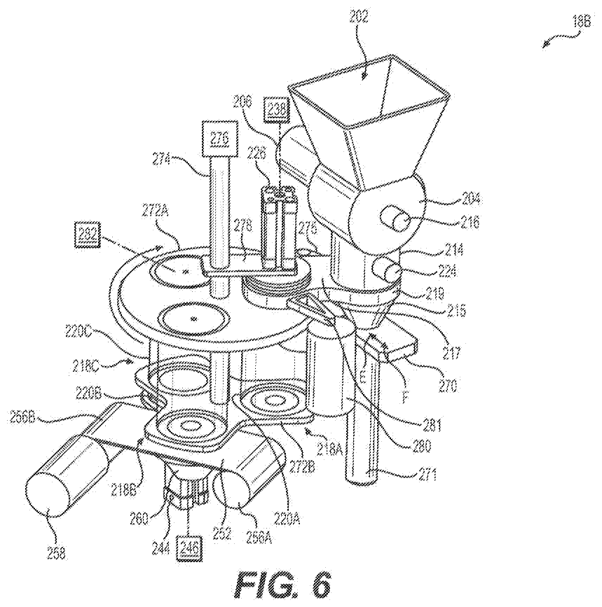

In some arrangements, soil preparation unit 18 may be serially aligned such that a single sample is prepared and delivered at a time to the soil analysis system 20. Such a system is depicted as soil preparation unit 18A and is illustrated in FIGS. 4 and 5. Alternatively, soil preparation unit 18 may be a parallel system in which multiple samples may be prepared, contemporaneously or simultaneously. Such a system is depicted as soil preparation unit 18B and is illustrated in FIG. 6. Each of soil preparation unit 18A and soil preparation unit 18B will be addressed in turn.

As shown in FIGS. 4 and 5, a serial soil preparation unit 18A may include a chute 102. Chute 102 may include an opening in an upper or top portion of soil preparation unit 18A. Chute 102 may be formed as a funnel having a wide mouth at one end which tapers towards a narrow opening at a second end. In such a manner, soil 32 samples collected via soil collection unit 14 may be delivered or guided into soil preparation unit 18A via chute 102. Soil preparation unit 18A may include a central chamber 104 in which a grinding rotor 108 (FIG. 5) may be mounted for rotation therein. Rotor 108 may be rotated via a motor 106 so as to grind the collected soil 32 sample. Rotor 108 may be any appropriate grinding apparatus such as, for example, a hammer mill or a disc mill.

A lower portion of central chamber 104 includes a sieve 110 (FIG. 5) through which the ground sample of soil 32 may be passed to separate course materials (e.g., stones, etc.). That is, sieve 110 may be a mesh filter including a plurality of pores 112 through which ground soil is passed. The size, shape, and arrangement of pores 112 may be arranged so as to pass a preferred size of ground soil 32 there through, while preventing larger, course components of the ground soil 32 from passing. The size, shape, and arrangement of pores 112 may be arranged for passing ground soil 32 through sieve 110 and into a measurement channel 114 at a selected rate (e.g., about 2.5 ml/s). For example, pores 112 may each have a diameter of between about 2 mm and about 20 mm. In some arrangements, for example, pores 112 may each have a diameter of about 10 mm. The separated course materials may be discarded via exit 116. For example, after the finely ground soil 32 has been passed through sieve 110, a vacuum source (not shown) connected to exit 116 may be activated for removing the separated course materials. Additionally, rather than the inclusion of a single sieve 110, as shown in FIG. 5), it is also contemplated that multiple sieves 110 may be serially aligned, one after the other, with decreasing pore 112 size. In this example, there may be multiple exits 116, one before each sieve 110 to remove waste.

As noted above, once passing through sieve 110, the ground soil 32 enters measurement channel 114. Measurement channel 114 may be arranged to separate a specific amount of soil 32 for analysis. Such an amount may be based on a volume of the soil 32 sample and/or a weight of the soil 32 sample. In other words, measurement channel 114 may prepare a dose of soil 32 for mixing with a liquid in mixing chamber 118. For example, measurement channel 114 may be sized to receive a specific volume of soil 32. By way of example only, such a sample may have a volume of about 32 ml. In order to measure the volume of the soil sample, measurement channel 114 may include a first gate or valve 120 (shown open in FIG. 5) and a second gate or valve 122 (shown closed in FIG. 5). Each of first valve 120 and second valve 122 may be a linear gate valve. To measure a dose of soil 32, second valve 122 is closed while first valve 120 remains open such that soil 32 passing from sieve 110 may be received within measurement channel 114. Once all of the soil sample has either been passed through sieve 110 or removed via exit 116, first valve 120 may be closed to separate, isolate and/or limit the amount of soil located within measurement channel 114. Any soil remaining above first valve 120 (e.g., closer to sieve 110) may be removed via exit 124. For example, a vacuum source (not shown) may be connected to exit 124 for removing excess soil. The excess soil removed from exit 124 may be delivered to soil analysis system 20 to measure the water content and pH value of the collected soil 32 sample, as will be discussed in further detail below.

In order to measure the weight of the soil sample, second valve 122 may have a load sensor/cell and/or scale (not shown) thereon. To measure a dose of soil 32, second valve 122 be closed while first valve 120 may remain open such that soil passing from sieve 110 may be received within measurement channel 114 and be measured via the load sensor/cell or scale. Once a desired weight (e.g., approximately 10 grams) of soil has been received on the load sensor/cell or scale, first valve 120 may be closed to limit the amount of soil 32 received within measurement channel 114. Once a proper amount or dose of soil 32 has been measured, second valve 122 may be opened to allow the dose of soil 32 to enter mixing chamber 118. The total time for a collected soil 32 sample to travel from chute 102 into mixing chamber 118 may be, for example, less than about 5 seconds.

Next, liquid may be added to mixing chamber 118 to mix with the soil therein. That is, a fluid may be delivered into mixing chamber 118 via a pump 126. As shown, pump 126 may be a piston pump having a piston 128 coupled to a piston rod 130 slideable with relative to a piston chamber 132. Upon actuation of piston rod 130 (e.g., via an electric or hydraulic motor, electro-magnet, or other actuating mechanism 138), fluid may be delivered into mixing chamber 118. For example, fluid may be delivered through nozzle 134 (FIG. 5) into mixing chamber 118. While pump 126 is illustrated as a piston pump, the disclosure is not so limited. Rather, pump 126 may include any appropriate pump such as, a peristaltic pump, or a dispenser valve apparatus. In any arrangement, pump 126 may deliver a specified volume of fluid. For example, pump 126 may deliver between about 160-250 ml of fluid. The total amount of time for the injected fluid to be delivered into mixing chamber 118 may be, for example, less than about 5 seconds.

The delivered fluid originates from a fluid supply (not shown) located on the vehicle 12, and may include a Morgan Solution. The Morgan Solution is a combination of water and a variety of chemicals, and may have the following composition: (0.72 N NaOAc (Sodium acetate trihydrate)+0.52 N CH₃COOH (glacial acetic acid) and distilled water). The Morgan Solution may be formed (e.g., mixed) prior to injection within mixing chamber 118. Alternatively, each constituent part (e.g., each chemical and water comprising Morgan Solution) may be individually injected into mixing chamber 118 and mixed therein. In such a scenario, multiple pumps, each arranged for injection of a single element of the Morgan Solution may be arranged for injection through nozzle 134 (or another entry port(s) into mixing chamber 118) without departing from the scope of this disclosure.

Once received within mixing chamber 118, the injected fluid (Morgan Solution) may mix with the dose of soil 32 to form a soil solution. In order to mix the soil and injected fluid, any appropriate mixing device may be used. For example, the entirety of soil preparation unit 18A may be shaken (e.g., via reciprocal or orbital motion of housing 52 or frame 53 (FIG. 1)) to mix the contents of mixing chamber 118. Alternatively, a paddle or other such member (not shown) may extend into mixing chamber 118 and may be rotated therein. In some arrangements, a magnet (not shown) may be arranged within mixing chamber 118. Additionally, a secondary magnet may be coupled to piston 140 of pump 144. Alternatively, piston 140 itself may be magnetized. In operation, piston rod 142 of pump 144 may be rotated (e.g., via a motor, electro-magnet, or other actuating mechanism 146). As the piston 140 rotates (e.g., at a speed of about 3 Hz, and for a period of time lasting between about 60 seconds and about 600 seconds), the magnet within mixing chamber 118 may likewise rotate due to magnetic interaction therebetween. Such rotation mixes the dose of soil 32 and injected fluid. Alternatively, piston 140 may be agitated (e.g., reciprocated and/or rotated) so as to mix soil 32 and Morgan solution.

Next, the mixed soil solution is filtered via a filter arrangement 150. Filter arrangement 150 includes a disposable filter sheet 152 moveably received between mixing chamber 118 and a head 162 (FIG. 5). Filter sheet 152 may be any appropriate filter material arranged to permit passage of liquid while preventing or blocking passage of any soil particulate within the mixed soil solution. For example, filter sheet 152 may be Whatman filter paper 3 which has a pore size of 6 μm (e.g., medium flow, thick filter paper). Filter sheet 152 may be moveable between a pair of spools 156A and 156B. Spools 156A and 1566 may rotate to position a new section or portion of filter sheet 152 between mixing chamber 118 and cylinder head 162 after each sample has been filtered. That is, spool 156A may rotate in one direction (e.g., clock-wise) so as to unwrap new filter sheet 152 portions while spool 1566 may rotate in the opposite direction (e.g., counter-clock-wise) so as to wrap used filter sheet 152 portions around spool 1566, as will be described in further detail below. As shown, at least one of spools 156A and 156B may be coupled to a motor 158 to facilitate rotation. For example, at least spool 1566 may be operably coupled to 1566 so as to pull used filter sheet 152 portions around spool 156B.

Head 162 may house a spring-loaded valve 155. For example, spring-loaded valve 155 is naturally closed (e.g., prevents passage of material there through) during filling and mixing in mixing chamber 118. Spring-loaded valve 155 may be opened via a pressure differential imparted by a pneumatic cylinder having an actuatable shaft 164 which may be operably coupled to an actuating mechanism 166 (e.g., a motor, electro-magnet, or other such device). That is, actuation of shaft 164 via actuating mechanism 166 may impart a pressure differential in head 162, to thereby open spring-loaded valve 155.

In order to urge the mixed soil solution through filter sheet 152 and into head 162, plunger 140 may be advanced towards filter sheet 152 via actuating mechanism 146 coupled to piston rod 142 so as to push the mixed soil solution. Once passed through filter sheet 152, the resulting filtered soil solution is a clear/colorless filtrate. That is, the resulting filtered soil solution has a zero-value color rating. In some arrangements, the resulting filtered soil solution may have an NTU rating in the range of between about 0 and about 20 NTU. The filtered soil solution may pass through spring-loaded valve 155 and exit soil preparation unit 18A and enter soil analysis system 20. For example, the filtered soil solution may exit soil preparation unit 18A via soil solution exit 160 on a lower or bottom portion of soil preparation unit 18A under the influence of gravity (or via a pump), and may be directed into soil analysis system 20 in any appropriate manner (e.g., ducts, tubes, etc.). The total time for a collected soil 32 sample to travel from chute 102 and through soil solution exit 160 may be, for example, less than about 15 seconds.

After each soil sample is processed though soil preparation unit 18A, soil preparation unit 18A may be cleaned prior to the receipt of a subsequent soil 32 sample through chute 102, to avoid cross-contamination between samples of soil 32. In order to clean soil preparation unit 18A, the soil preparation unit 18A may be rinsed with water (e.g., optionally warm water). For example, warm water may be introduced through chute 102. In some examples, the warm water may be injected under a high pressure, e.g., between about 2 bar and about 15 bar, via any appropriate manner. The water may travel through soil preparation unit 18A, rinsing grinding rotor 108, passing through sieve 110, into measurement channel 114, into mixing chamber 118, and exit through a cleaning port 168 (FIG. 5). One or more additional rinses with a variety of cleaning agents may be passed through soil preparation unit 18A in a similar manner. Such additional rinses may include an aluminum chloride solution, and deionized water. For example, following the warm water, a first aluminum chloride solution rinse may be passed through the soil preparation unit 18A, and two additional rinses of deionized water may be passed through the soil preparation unit 18A, thereafter. Optionally, any one or more of these additional rinses may be pressurized in any appropriate manner. Following any or all rinses, soil preparation unit 18A may be dried. For example, a blower or other such device (not shown) may be attached to soil preparation unit 18A to forcibly dry soil preparation unit 18A. Optionally, a blower may be positioned on one of the sides of central chamber 104, so that central chamber 104 can be filled with air which can exit at exit 116 and/or pass into mixing chamber 118 and out through cleaning port 168. Alternatively, soil preparation unit 18A may be left to air-dry. In some arrangements, soil preparation unit 18A need not be dried prior to receiving a subsequent soil sample. The total time for cleaning soil preparation unit 18A may be, for example, less than about 120 seconds.

As noted above, in some arrangements, soil preparation unit 18 may be a parallel or batch system in which multiple samples of soil 32 may be prepared contemporaneously or simultaneously. Such a system is depicted as soil preparation unit 18B and is illustrated in FIG. 6. Soil preparation unit 18B is similar in construction and purpose to soil preparation unit 18A, and as such, like components will be labeled the same, plus 100.

As shown in FIG. 6, a parallel/batch soil preparation unit 18B includes a chute 202 leading towards a central chamber 204. Similarly to soil preparation unit 18A, central chamber 204 may include a grinding rotor (not shown) mounted for rotation therein via motor 206. A lower portion of central chamber 204 includes a sieve (not shown) through which the ground soil 32 sample may be passed to separate course materials (e.g., stones, etc.). The separated course materials may be discarded via exit 216. For example, a vacuum source (not shown) may be connected to exit 216 for removing the separated course materials.

Unlike soil preparation unit 18A, however, parallel/batch soil preparation unit 18B includes three stages or stations, each stage identified for a specific functionality. Alternatively, more or less than three stations may be employed. Within each station, a tube or chamber (e.g., 220A-220C) is disposed. Chambers 220A-220C are mounted for rotation between two supports or plates 272A and 272B so as to move sequentially from one station to the next. The three stations include filling station 218A, mixing/filtering station 218B, and cleaning station 218C, as will be described in further detail below. Each of first plate 272A and second plate 272B may be rotatably supported by a shaft 274. That is, each of first plate 272A and 272B may rotate about shaft 274 via revolver actuator 275, as will be described in further detail below. Additionally, shaft 274 may be coupled to support 278. As such, rotation of shaft 274 may result in likewise rotation of support 278, which is coupled to pump 226 (similar to pump 126 in FIG. 5).

Once passing through the sieve, soil 32 enters measurement chamber 214. Measurement chamber 214 may be arranged to separate a specific amount of soil 32 for analysis (e.g., by weight or volume) in any appropriate manner. For example, in some arrangements, a press, stamp, or other such mechanism (not shown) may be arranged within measurement chamber 214 to cut or otherwise divide a soil 32 sample from soil 32. Once measured, the dose of soil 32 may be moved into first chamber 220A located at filling station 218A, in any appropriate manner. For example, the dose of 32 may be moved along an inner chamber of connection 217 via a belt or conveyer 219 moveable relative to connection 217 via an actuator 280 including an arm 281. That is, in some examples, rotation of 280 may rotate arm 281 to push conveyer 219 towards filling station 218A to direct the dose of soil 32 into first chamber 220A. Alternatively, the dose of soil 32 may be delivered into first chamber 220A in any appropriate manner.

Additionally, after the appropriate dose of soil 32 has been collected and moved to first chamber 220A, at least a portion of any excess soil 32 may be removed via exit 224. For example, a vacuum source (not shown) may be connected to exit 224 for removing excess soil 32. The excess soil 32 removed from exit 224 may be delivered to soil analysis system 20 to measure the water content and pH value of the collected soil 32 sample, as will be described in further detail below. Further, any additional excess soil 32 not moved into first chamber 220A, or removed via exit 224, may be discarded into waste chamber 271. For example, a slider 270 may be moved in the direction F to facilitate delivery of excess soil from measurement chamber 214 into waste chamber 271 via guide 215. Once any excess soil 32 has been moved to waste chamber 271, slider 270 may be moved in the direction E to close measurement chamber 214 and guide 215.

Once the soil 32 has been collected and transferred to filling station 218A, revolver actuator 275 may be actuated to rotate plates 272A and 272B to move first chamber 220A into mixing/filtering station 2186. Additionally, upon rotating the plates 272A and 272B about 120°, chambers 220B and 220C will be located in the cleaning station 218C and filling station 218A, respectively. Simultaneously, or before or after such movement of plates 272A and 272B, shaft 274 may be rotated via motor 276 to move pump 226 over second station 218B. Next, a fluid may be delivered into chamber 220A (now in mixing/filling station 218B) via pump 226. Pump 226 may be a piston pump having a piston (not shown) coupled to a piston rod (not shown) slideable relative to a piston chamber (not shown). Upon actuation of the piston rod (e.g., via a motor, electro-magnet, or other actuating mechanism 238), fluid may be delivered into chamber 220A. While pump 226 is illustrated and described as a piston pump, the disclosure is not so limited. Rather, pump 226 may include any appropriate pump such as, a peristaltic pump, or a dispenser valve apparatus. In any arrangement, pump 226 delivers a specified volume of fluid. For example, pump 226 delivers between about 160-250 ml of fluid. The delivered fluid may include a Morgan Solution.

In order to mix the soil and fluid within filling station 218A, revolver actuator 275 may rotate, shake, or otherwise disturb the contents of mixing/filtering station 2186. Revolver actuator 275 may be actuated in any appropriate fashion so as to rotate back and forth plates 272A and/or 272B so as to mix/shake the contents of chamber 220A in mixing/filtering station 218B. As shown, mixing/filtering station 218B includes a disposable filter sheet 252 moveably received between a chamber (e.g., one of chambers 220A-220C) and an evacuation pump 244. Filter sheet 252 may moveable between a pair of spools 256A and 256B. Spools 256A and 256B may rotate to position a new section or portion of filter sheet 252 between mixing chamber 118 and evacuation pump 244 after each sample of soil 32 has been filtered. That is, spool 256A may rotate in one direction (e.g., counter-clock-wise) so as to unwrap new filter sheet 252 portions while spool 256B rotates in the same direction (e.g., counter-clock-wise) so as to wrap used filter sheet 252 portions around spool 256B. As shown, at least one of spools 256A and 256B may be coupled to a motor 258 to facilitate rotation. For example, at least spool 256B may be operably coupled to motor 258 so as to pull used filter sheet 252 portions around spool 256B.

Evacuation pump 244 may draw or suck the mixed soil solution through filter 252 to separate any remaining soil particulate via motor or actuator 246, thus resulting in a filtered soil solution. The filtered soil solution may exit mixing/filtering stage 218B through soil solution exit 260 and be directed towards soil analysis system 20. Additionally, once filtered, revolver actuator 275 may be activated to thereby rotate plates 272A and 272B. Upon rotating the shaft 274 about 120°, the now substantially empty chamber 220A may be positioned within cleaning station 218C (and chambers 220B and 220C will be located in the mixing/filtering station 218B and filling station 218A, respectively). As shown, cleaning station 218C may be operably coupled to a cleaning system 282. Cleaning system 282 may include any appropriate number of tanks or pressurizers to deliver one or more rinses (e.g., warm water, aluminum chloride solution, and deionized water) and/or a blower or other such device to forcibly dry chamber 220A. It is understood, that as chamber 220A is rotated between filling station 218A, mixing/filtering station 218B, and cleaning station 218C, each of chambers 220B and 220C will be likewise rotated. In such a manner, multiple batches of soil 32 sample may be prepared contemporaneously with one another.

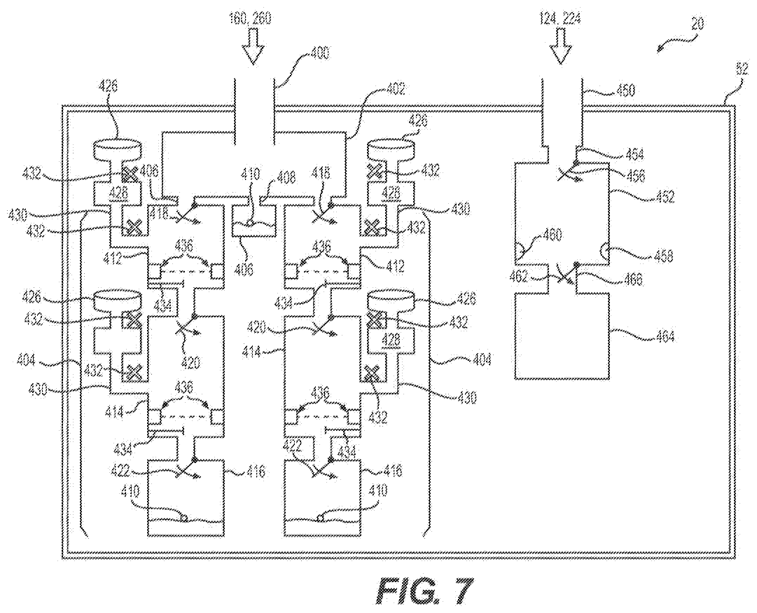

Once the collected soil sample has been prepared into a color-less filtered soil solution via either soil preparation unit 18A or soil preparation unit 18B, the filtered soil solution may be delivered from soil solution exit 160, 260 into the soil analysis system 20. As shown in FIG. 7, soil analysis system 20 may include an entry port 400. Upon entry through port 400, the color-less filtered soil solution may be received within a manifold 402. Manifold 402 may divide the colorless filtered soil solution into one or more sub-samples, each of which may directed to a sub-sample path 404 via a duct 406. For example, the colorless filtered soil solution may be divided into any appropriate number of sub-samples, such as, e.g., eight sub-samples. The number of sub-samples into which the colorless filtered soil solution is divided may correlate to the number of specific chemical nutrients to be measured. For instance, soil analysis system 20 may be equipped to test eight nutrients. That is, soil analysis system 20 may be equipped to measure one or more of iron (specifically: ferric Fe2+ferrous iron Fe³⁺), Calcium Ca²⁺, magnesium Mg²⁺, sulfur (specifically: sulfate sulfur SO₄²⁻), potassium (specifically: potassium oxide K⁺), nitrogen (specifically: nitrate nitrogen NO₃), phosphorous (specifically: phosphorous pentoxide P₂O₅), manganese Mn(OH)₂, and aluminum Al. While only two sub-sample paths 404 are shown in FIG. 7 for the sake of brevity, it is understood that each of the eight sub-sample paths 404 would be similarly arranged except for the specific reagent applied, as will be discussed in further detail below. Further, it is understood that each of the sub-samples may travel through an appropriate sub-sample path 404, contemporaneously. That is, a first sub-sample may be conveyed through a first sub-sample path 404 while a second sub-sample may be conveyed through a second sub-sample path 404, and so on, at the same time. As each sub-sample may be analyzed in parallel and at the same time, the total time required to analyze all eight of the identified nutrients may be reduced.

Each sub-sample may have a specified volume. For example, each sub-sample may be between about 2 ml and about 10 ml. Any excess colorless filtered soil solution not divided into a sub-sample path 404, may be discarded and collected in an excess tank 406 via port 408. Excess tank 406 may include a sensor (e.g., float sensor 410) to measure the fill level of excess tank 406, and deliver an appropriate signal to controller 50, as will be described in further detail below.

As shown in FIG. 7, each sub-sample path 404 may have a plurality of serially aligned chambers 412, 414, and 416 downstream of duct 406. Each of the plurality of chamber may include an upstream entry valve. For example, a first chamber 412 may include a first upstream entry valve 418, a second chamber 414 may include a second upstream entry valve 420, and a third chamber 416 may include a third upstream entry valve 422.

Additionally, each of first chamber 412 and second chamber 414, of each sub-sample path 404, may be fluidly coupled to a reagent tank 426 via a dosing tank 428 and a duct 430. Tanks 426 maybe modular and can be easily removed and replaced as cassette or cartridges. Reagent tanks 426 of a first sub-sample path 404 may include a common reagent or chemical therein. That is, each of the reagent tank 426 coupled to first chamber 412 and the reagent tank 426 coupled to second chamber 414 in a first sub-sample path 404 may contain the same reagent or chemical. However, reagent tanks 426 of a second sub-sample path 404 may contain a different reagent than that of the first sub-sample path 404. In other words, each sub-sample path 404 may contain two reagent tanks 426 having the same reagent therein, but the reagent in the reagent tanks 426 of each sub-sample path 404 will be different than any other sub-sample path 404. Reagents will be added to colorless solution. Once added, a reaction will occur which may lead to a colored solution which may be tested for soil analysis. Exemplary reagents may include one or more of the following: reagents ferric iron-hydrochloric acid, distilled water, potassium sulfocyanate; reagents ferrous iron-hydrochloric acid, distilled water, potassium ferricyanide; reagent calcium-sodium oxalate; reagent magnesium-para-nitrobenzene azo-resorcinol, sodium hydroxide, titan yellow, methyl alcohol, sodium hydroxide, distilled water; reagents sulfate sulfur-barium chloride and distilled water; reagents potassium oxide-cobalt nitrate, sodium nitrite, glacial acetic water, iso-propyl alcohol, and distilled water; reagents nitrate nitrogen-diphenylamine, concentrated sulfuric acid; reagents phosphours pentaoxide-sodium molybdate, acetic acid, universal extraction solution, stannous oxalate and distilled water; reagents manganese-benzidine, glacial acetic acid, sodium hydroxide, saturated solution of potassium periodate in universal soil extracting solution and distilled water.

Each dosing tank 428 may house or contain a preferred volume of reagent. As such, each dosing tank 428 may limit an amount of reagent to be delivered into a respective one of first tank 412 and 414. Dosing tank 428 may measure an amount in any appropriate manner such as, for example, via one or more valves (not shown) which may remain closed until dosing tank 428 is filled, and subsequently, may be opened to deliver the specified amount (e.g., volume) of reagent into one of first chamber 412 and second chamber 414, via duct 430. Further, each duct 430 may include a plurality of flow rate controls 432 to adjust a flow rate of reagent entering one of first chamber 412 and second chamber 414, or to adjust the flow rate of reagent entering dosing tank 428. Further, as shown, each first chamber 412 and second chamber 414 of each sub-sample path 404 includes a mixer 434 (e.g., a rotatable paddle) therein. Mixer 434 may be actuated to mix a sub-sample with a dose of reagent, as will be described in further detail below.

When activated (e.g., via a user interface or during the course of executing a pre-defined set of commands), first upstream entry valve 418 may open, thereby allowing a sub-sample to enter first chamber 412 of each sub-sample path 404, while second and third upstream entry valves 420 and 422 remain closed. Accordingly, the sub-sample is received and maintained within first chamber 412. Next, a specified dose of reagent, as measured by dosing tank 428 is added to first chamber 412. Then, photometric unit 436 (or any other sensor) may be used to determine whether the dose of reagent and the sub-sample were received within first chamber 412. The mixer 434 may then mix the combined solution.

Once photometric unit 436 of first chamber 412 has determined that the dose of reagent and soil 32 sub-sample has been added to first chamber 412, the reagent-mixed sub-sample exits first chamber 412. At this stage, second upstream entry valve 420 of second chamber 414 is opened to allow the reagent-mixed sub-sample to be received therein. Here, a similar process is repeated such that a dose of the same reagent is delivered to second chamber 414 via reagent tank 426, dosing tank 428, and duct 430. The reagent-mixed sub-sample undergoes a second stage of mixing via mixer 434, and photometric unit 436 may detect a nutrient quantity of the reagent-mixed sub-sample. Here, photometric unit 436 within second chamber 414 may include a pair of optical sensors/receivers (e.g., an optical source/light transmitter and an optical source/light receiver) that communicate to determine a value indicative of a specific nutrient. For example, the reagent delivered to second chamber 412 and 414 in a first sub-sample path 404 may result in a red combined solution. Photometric unit 436 of the second chamber 414 (and optionally the first chamber 412) may be used to measure a depth of color of the red combined solution, which is indicative of an amount of a specific nutrient (e.g., ferrous iron, with a measurement frequency of 523 nm). It is understood that each photometric unit 436 of soil analysis system 20 is uniquely configured for testing each specified nutrient. That is, each photometric unit 436 is tailored such that the optical source/light transmitter transmits an optical source of a specified wavelength, and the optical source/light receiver receives optical energy/light having a specified wavelength. The nutrient value is determined by a unique calibration curve which correlates a photometric reading from photometric unit 436 and nutrient concentration. It is important to note, however, that not all reagents will result in a colored combined solution. Rather, some reagents, when delivered and mixed with a sub-sample of solution, may result in a milky, or opaque combined solution. In such cases, photometric unit 436 may be used to measure the degree of turbidity of the combined solution, which is indicative of an amount of a specific nutrient (e.g., calcium).

Once testing is complete, the reagent-mixed sub-sample may be discarded to third chamber 416 for storage, and subsequent disposal. Further, while each sub-sample path 404 is shown to include an individual third chamber 416, in some arrangements, each sub-sample path 404 may deliver to a common third chamber 416 or excess tank. Third chamber 416 may include a sensor (e.g., float sensor 410) to measure the fill level of third chamber 416, and deliver an appropriate signal to controller 50, as will be described in further detail below

As noted above, excess soil removed from exit 124 or 224 may be delivered to soil analysis system 20 to measure the water content and pH value of the collected soil sample. For example, as shown in FIG. 7, the excess soil may enter soil analysis system 20 via entry port 450. From there, the excess soil may enter first chamber 452 via a duct 454 and an entry valve 456. As shown, first chamber 452 includes a water probe 458 and a pH sensor 460. Water probe 458 may be any commercially available water probe designed to measure a water content of a sample. Likewise, pH sensor 460 may be any commercially available pH measurement device designed to determine the pH value of a sample. Once testing is complete, the excess soil may be delivered to a second chamber 464 via entry valve 462 and duct 466. Second chamber 464 may receive the excess soil for storage, and subsequent disposal. It is understood that any of the excess or waste tanks discussed herein may include a neutralization agent to make the system 10 waste less hazardous, and may be removed/replaced whenever full or desired. Once one or more of the nutrients, the water content, and/or the pH value has been determined, soil analysis system 20 may be cleaned. That is, similar to soil preparation unit 18, one or more rinses (e.g., warm water, aluminum chloride solution, and deionized water) may be passed through the soil analysis system. Optionally, a blower, fan, or other such device may be coupled to soil analysis system 20 to forcibly dry one or more components therein.

FIG. 8 illustrates various communication signals between components of system 10. As noted above, controller 50 may send and receive signals from central platform 24. For example, upon determining a need or desire to measure and/or fertilize soil 32 (FIG. 1), central platform 24 may deliver a signal to controller 50 on vehicle 12 to initiate operation. As noted above, central platform 24 may deliver a pre-determined track or course of travel to vehicle 12 via controller 50. Additionally, vehicle 12 may employ one or more of sensors 46 and GPS tracker 48 to determine its own preferred course of travel, or modify a pre-determined track or course of travel dynamically, and deliver such a signal to central platform 24 via controller 50.

Once initiated, vehicle 12 may drive to a selected location. Once in position, soil collection unit 14 may be actuated to collect a sample of soil 32, as discussed above. Accordingly, controller 50 may communicate with one or more components of soil collection unit 14 (e.g., motor 70) to facilitate raising, lowering, and/or rotating drill 60, collecting a sample of soil 32, and autonomous delivery of the collected sample of soil 32 to soil preparation unit 18 of onboard analysis unit 16.

Once a sample of soil 32 is received by soil preparation unit 18, temperature sensor 67 may communicate a signal indicative of onboard analysis unit 16 to controller 50. Controller 50 may analyze the received signal to ensure the temperature does not fall outside of an acceptable range of temperature value to enable proper chemical reaction between soil 32 and the injected Morgan Solution, as described above. If the temperature signal indicates that the temperature is outside of the acceptable range of temperature values, the controller 50 may deliver a warning or signal via interface 54 and/or to central platform 24. Additionally, if the temperature signal indicates that the temperature is too low, controller may activate a heater (not shown) or other such device to raise the temperature of onboard analysis unit 16 so that it returns to an acceptable range of temperature.

Additionally, as shown in FIG. 8, controller 50 may communicate with soil analysis system 20. That is, one or more of water probe 458, pH sensor 460, photometric units 436, and float sensors 410 may send a signal indicative of a sensed condition to control 50. Upon receipt of such a signal, controller 50 may make a recommendation or send an alert to a user via interface 54 and/or to central platform 24. Additionally, signals output from water probe 458, pH sensor 460, photometric units 436 may be delivered to controller 50 and central platform 24 for determination of a recommendation on fertilization. For example, controller 50 may collect these outputs, compare the outputs to data stored in a memory or storage 23, execute any appropriate analysis software 27 and communicate a result to central platform 24. Additionally, central platform 24 may correlate the received signals with weather conditions, soil and plant types, as well as field locations, in order to generate an optimal recommendation for subsequent fertilization and/or treatment. For example, central platform 24 may determine that the sampled soil 32 is deficient in any one or more of the measured nutrients, water content, or falls outside of an ideal pH value range for a specific plant.

Once controller 50 has made such a determination, controller 50 may communicate with fertilization/irrigation unit 22 to deliver a tailored cocktail of nutrients to soil 32. These fertilizing nutrients may be stored onboard the same vehicle 12, or on a second separate vehicle 12 of system 10. Each nutrient may be contained within a refillable tank or supply of nutrients 15A-15H, and irrigation fluid may be contained in a refillable tank of irrigation fluid (e.g., water) 15I.

The nutrient(s) and/or irrigation fluid may be injected via a plunger pump, or the like. For example, each supply 15A-15I may be coupled to a tube (not shown) connected to an appropriate pump or suction source. The selected nutrients may be sucked or pulled into the tubes, where they may be delivered individually under pressure into soil 32, or may be mixed prior to delivery under pressure into soil 32. Additionally, fertilization/irrigation unit 22 may have a predetermined flow rate monitor (not shown) which may be used for all nutrient and irrigation injections. Based on an output of soil analysis unit 20, central platform 24 may advise the farmer/owner/operator what nutrients and what amounts are necessary to be injected or are lacking. If the operator accepts a prompt via central platform 24, the nutrient cocktail will be added in the amount determined by computer platform 24 via fertilization/irrigation unit 22. For example, if central platform 24 determines that a specific sample of soil 32 lacks three out of the eight tested nutrients, controller 50 may instruct the fertilization/irrigation unit 22 to inject only those nutrients to the soil 32. Delivery of the selected nutrients may occur in any appropriate manner such as, for example, spraying or injecting the nutrients into soil 32. Alternatively, delivery of the selected nutrients may include implantation of solid pellets or powder either by gravity feed or injecting the solid nutrient pellets or powder into the soil 32 at specified depths for slow-release nutrients. In some arrangements in which drill 60 includes a hollow coring drill bit, injection of the selected nutrients may occur via the internal central hollow passage (not shown) of drill 60. In either way, fertilization of soil 32 is specifically tailored (e.g., optimized) to address the deficiencies found in soil 32, and avoid over fertilization of those nutrients deemed to be sufficient. Accordingly, over generalization of a large field of soil 32, ground water contamination, and eutrophication may be avoided. Additionally, fertilization/irrigation unit 22 may communicate with controller 50 to deliver one or more signals indicative of a remaining volume within each nutrient source tank 15A-15H or irrigation fluid tank 15I. If one or more of the nutrient or irrigation supplies is found to be empty, or below a threshold value, controller 50 may deliver a signal via one or both of interface 54 and central platform 24 to indicate a refill is necessary. Additionally, as controller 50 may communicate with central platform 24, fertilization of soil 32 may happen in real-time, avoid delays or lag in fertilization. In addition to real-time fertilization and/or irrigation of soil 32, upon receipt of the information to central platform 24, central platform 24 may execute one or more processes (e.g., via a processor) to mine the collected data and prepare a recommendation to add or remove planned sampling and/or analysis activities in real-time.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

1. A system, comprising:
a vehicle including a controller; and
an onboard soil analysis unit including a soil preparation unit and a soil analysis system for real-time analysis.

2. The system of claim 1, wherein the vehicle is a first vehicle, the system further including at least one additional vehicle having an onboard analysis unit.

3. The system of claim 1, further including a soil collection unit having a drill and/or a soil capturing device.

4. The system of claim 1, further including a fertilization and/or irrigation unit, wherein the fertilization and/or irrigation unit includes a plurality of modular nutrient tanks or one or more containers for solid pellets or powders.

5. The system of claim 1, further including a central platform, the central platform being wirelessly communicable with the controller of the vehicle.

6. The system of claim 1, wherein the system includes a plurality of vehicles, each vehicle of the plurality of vehicles being in communication with a central platform and configured for performing one or more of soil sampling, soil analysis, soil fertilization, and soil irrigation.

7. A method, comprising:
collecting a sample of soil via a soil collection unit;
mixing the soil with a liquid to form a colorless solution;
dividing the colorless solution into one or more sub-samples and adding a specific reagent to each sub-sample;
analyzing each sub-sample to determine a nutrient value via a soil analysis unit in real-time.

8. The method of claim 7, wherein dividing the colorless solution into one or more sub-samples includes dividing the colorless solution into eight sub-samples.

9. The method of claim 7, wherein analyzing each sub-sample includes taking a photometric measurement of the sub-sample.

10. The method of claim 9, further including, wirelessly delivering the photometric measurement to a central platform and delivering a recommendation to a user.

11. The method of claim 10, further including delivering a tailored fertilizer to a field based at least on the photometric measurement.

12. The method of claim 11, wherein analyzing each sub-sample and delivering the tailored fertilizer is done in real-time.

13. The method of claim 7, further including maneuvering a vehicle coupled to the soil analysis unit autonomously.

14. The method of claim 7, wherein collecting a sample of soil via a soil collection unit includes collecting a plurality of samples at varying depths.

15. The method of claim 7, wherein mixing the soil with a liquid includes mixing the soil with a Morgan Solution.

16. The method of claim 7, further including analyzing the data collected via the soil analysis unit, correlating the data with stored data on a central platform, and delivering a recommendation to user.

17. An analysis unit, comprising:
a soil preparation unit, including
a mixing chamber;
a fluid pump;
a source of Morgan Solution; and
a soil analysis system including a plurality of reagent tanks;
wherein the analysis unit is coupled to a vehicle.

18. The analysis unit of claim 17, wherein the soil analysis system further includes a plurality of sub-sample paths.

19. The analysis unit of claim 18, wherein the soil analysis system includes at least eight sub-sample paths.

20. The analysis unit of the claim 17, wherein the soil preparation unit further includes a mixer configured to mix soil and Morgan Solution to form a colorless solution.
